(19) United States
(12) Patent Application Publication
(10) Pub. No.: US 2007/0112290 A1
(43) Pub. Date: May 17, 2007

(54) Artificial auditory tube

(76) Inventor: Masahiro Morita, Osaka (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/577,998

(22) PCT Filed: Oct. 28, 2004
(86) PCT No.: PCT/JP04/15995

§ 371 Date: May 3, 2006

(30) Foreign Application Priority Data
Nov. 5, 2003 (JP) 2003-375097

(51) Int. Cl. A61F 11/00

(52) U.S. Cl. 604/8

(57) ABSTRACT

Disclosed is an artificial auditory tube that is a therapeutic instrument useful for the treatment of eustachian tube dysfunction including stenosis of the eustachian tube (occlusion of the eustachian tube), patulous eustachian tube, closure defect of the eustachian tube, and “floppy tube”. The artificial auditory tube consists of a tubular body having a proximal end to be placed protruding out of the tympanic membrane within the external auditory canal and having a distal end to be inserted from the tympanic cavity into the eustachian tube and placed through the isthmus and facing the inside of the cartilaginous eustachian tube, the distal end and the proximal end communicating with each other through an internal cavity extending in the axial direction of the tubular body, the internal cavity opening to the outside through a first opening located in the distal end portion, and the external diameter falling in the range of from 0.35 to 3.0 mm, wherein the artificial auditory tube includes a second opening defined in the wall of the tubular body and positioned in the region ranging from 9 to 30 mm away from the distal end and communicating with the internal cavity of the tubular body.

### TECHNICAL FIELD

The present invention relates to an artificial auditory tube to be inserted in the eustachian tube through the tympanic membrane.

### BACKGROUND ART

The ear consists of the external ear, middle ear and inner ear. The external ear and the middle ear are partitioned with the tympanic membrane, which is located at the inner end of the external auditory canal. The middle ear consists of the tympanic cavity (middle ear cavity), i.e., the space enclosing in it auditory ossicles (the malleus, incus and stapes) that bridge between the tympanic membrane and the oval window, which leads to the vestibule of the inner ear, and the eustachian tube extending from the tympanic cavity and opens at the pharynx. The eustachian tube starts from the vestibule of the tympanic cavity (the tympanic opening of the eustachian tube), extends obliquely in the direction running from the upper-rear-lateral side to the lower-frontal-medial side, and opens in the lateral wall of the pharynx (the pharyngeal orifice). The full length of the eustachian tube is around 33 mm, with its superior approximate one third running through the temporal bone and its inferior approximate two thirds being wrapped with a cartilage. The osseous eustachian tube, after once having somewhat broadened passing through the narrowed tympanic opening of the eustachian tube, tapers and becomes the narrowest at the inlet to the cartilaginous portion (the isthmus of the eustachian tube), where it is normally closed. Down below the isthmus, the eustachian tube gradually broadens and opens, like a horn, as the pharyngeal orifice. One of the functions of the eustachian tube is a ventilatory function. This function is further divided into active and passive ones; in the former, the lumen of the isthmus temporarily opens during yawning or swallowing, with which the inferior wall of the cartilaginous portion is pulled downward by the contraction of the tensor veli palatini muscle, and air influx thus follows from the pharynx into the tympanic cavity, and in the latter, ventilation is caused passively by changes in the external air pressure. In the normal ear, the tympanic cavity pressure is maintained equivalent to the ambient pressure by the ventilatory function, in particular, by the active ventilatory function of the eustachian tube. The eustachian tube also has a drainage function, by which the secretions in the middle ear are eliminated out into the pharynx. There are conditions where these functions are disturbed, i.e., eustachian tube dysfunctions, such as stenosis of the eustachian tube (occlusion of the eustachian tube), patulous eustachian tube, and closure defect of the eustachian tube.

Stenosis of the eustachian tube is a condition in which the opening of the eustachian tube that should occur during swallowing or yawning is hindered by some causes and thus the ventilation of the middle ear through the eustachian tube is disturbed. Such causes include organic stenosis of the eustachian tube as a result of, e.g., inflammation in the epipharynx, and functional stenosis due to dysfunctioning of the muscle responsible for opening the eustachian tube (tensor veli palatini muscle), as observed in cleft palate. As the oxygen in the tympanic chamber is absorbed by the surrounding mucosa, when the ventilation of the middle ear is hindered due to stenosis of the eustachian tube, the tympanic chamber pressure turns negative and thus the tympanic membrane becomes retracted. This leads to symptoms such as a feeling of fullness in the ear, hearing loss, and autophony. Persistent stenosis of the eustachian tube will sometimes lead to otitis media with effusion. This disorder, in which collection of effusion is present in the middle ear cavity as a result of persistent negative pressure in the tympanic cavity, not only causes conductive hearing loss and a feeling of fullness in the ear due to the effusion that fills the tympanic cavity, but also render the patient susceptible to recurrent acute otitis media. Further, a situation in which the tympanic cavity is kept under negative pressure chronically and irreversibly could cause adhesive otitis media, a very refractory disease in which the tympanic membrane adheres to the wall of the tympanic cavity, or otitis media with cholesteatoma, a disease in which the corneal squamous epithelium of the tympanic membrane grows in the middle ear cavity, which originally is free of epithelium, corroding, along with it, the surrounding bone.

To treat stenosis of the eustachian tube, a so called eustachian tube aeration technique is commonly applied, in which aeration is performed through a catheter inserted into the pharyngeal orifice of the eustachian tube. Other methods for treatment include infusion of a steroid hormone into the eustachian tube from the pharyngeal or tympanic cavity side, or subcutaneous injection of a steroid hormone around the pharyngeal orifice of the eustachian tube. However, the efficacy of these methods has not yet been objectively verified. As conservative treatments by medication, there are conducted systemic administration of antiinflammatory enzyme preparations or anti-allergic preparations, and nasal instillation of steroidal preparations. However, there still is a problem that they require a long term administration of such preparations, and they are only poorly effective in many of the moderate or severer cases. In cases where no significant effect is observed by medication, placement of a tympanostomy tube is also conducted in order to attain ventilation of the tympanic cavity. A tympanostomy tube is a tube designed to be fit in the perforation made in the tympanic membrane, and those tubes varying in size and shape are commercially available. One of such commonly used tympanostomy tubes is a tube of approximately 3 mm in length and of constricted diameter in its central area. A tympanostomy tube provides ventilation of tympanic cavity. It, however, does not serve to improve the stenosis of the eustachian tube, and, therefore, ventilatory and drainage function by way of the eustachian tube will not be restored by it. In addition, a tympanostomy tube spontaneously comes off, usually in several months to one year, as the perforation formed in the tympanic membrane heals, making it necessary to place a new tympanostomy tube again to attain ventilation, if the stenosis of the eustachian tube is not improved. For cases that resist medication, a treatment technique has recently been developed by which the mucous membranes in the eustachian tube is cauterized using a laser (carbon dioxide laser, KTP laser), approaching from the pharyngeal orifice of the eustachian tube. However, it has not become a popular technique, for, while the influence is not well known on the surrounding tissues when a deep zone close to the isthmus is cauterized, one is required, for conducting eustachian tube cauterization, to have sufficient knowledge of relevant anatomy and to have mastered a highly sophisticated technique of the surgical operation.

“Patulous eustachian tube” represents a condition where the eustachian tube is always open. Subjective symptoms of the patient include autophony, which is caused by transmission of patient's own voice to the middle ear through the eustachian tube, hearing the patient's own breathing, a feeling of fullness in the ear, and, in some cases, vertigo. The tympanic membrane of the patient is found normal but observed to move back and forth as he breathes. The causes of patulous eustachian tube include atrophy of the nasal mucosa due to aging or neurological disorders, shrinkage of the mucosa around the eustachian tube as a result of weight loss, scars left after adenoid operation, and so on. In the majority of cases, however, the cause remains unknown.

Examples of medications for patulous eustachian tube include Bezold's method, in which a mixture powder of boric acid and salicylic acid is insufflated into the eustachian tube using eustachian tube catheter, infusion of a gelatin sponge solution into the eustachian tube lumen, etc.; and examples of surgical treatment include injection of liquid silicone, cauterization of otic mucosa, transposition of the tensor veli palatini muscle, implanting of a piece of cartilage or adipose tissue around the eustachian tube, infusion of a collagen solution, etc. It has been the problem that while medication for patulous eustachian tube requires a long term, continuous treatment, surgical treatment does not exhibit sufficient efficacy. Meanwhile, there has been proposed a trans-tympanic plug of a tapered, flat shape as a device for treating patulous eustachian tube and closure defect of the eustachian tube, which device is placed in the eustachian tube's lumen up to 5-15 mm deep from the tympanic membrane (see Patent Document 1). This device, however, can not be applied to treat stenosis of the eustachian tube, for it works by clogging the cross section of the eustachian tube.

A so called “floppy tube” has recently been drawing attention for its implication in the eustachian tube function. A “floppy tube” represents a eustachian tube that is easily occluded and also easily kept open. In this, yawning or swallowing makes the eustachian tube kept open, thereby causing autophony and a feeling of fullness in the ear. To remove such unpleasant symptoms, the patient tends to repeat sniffing unintentionally (by this, the tympanic cavity pressure becomes negative and the eustachian tube thereby closed). When this repeated sniffing becomes a habit and the tympanic cavity thus gets placed at chronic and irreversible negative pressures, the condition could cause otitis media with effusion, adhesive otitis media, and otitis media with cholesteatoma, as described above in relation to stenosis of the eustachian tube.

As noted above, dysfunction of the eustachian tube causes a variety of middle ear disorders, and thus there have been needs for a reliable method for treatment that enables effectively and easily treat stenosis of the eustachian tube, patulous eustachian tube, or floppy tube, which develops both occlusion and lasting patency, and that can be used to treat adhesive otitis media as well as to prevent adhesion of the tympanic membrane after surgical operation of otitis media with effusion and to prevent the recurrence of otitis media with cholesteatoma.

[Patent Document 1] Japanese Patent Application Publication No. 2002-224157

### DISCLOSURE OF INVENTION

### The Problem to be Solved by the Invention

Against the above-mentioned background, the objective of the present invention is to provide an instrument useful to treat eustachian tube dysfunctions including stenosis of the eustachian tube (occlusion of the eustachian tube), patulous eustachian tube, closure defect of the eustachian tube, and “floppy tube”.

### MEANS TO SOLVE THE PROBLEM

The present inventor discovered that a tube of a certain shape having an ventilation opening in its wall serves to very effectively treat eustachian tube dysfunctions and thus can be used as an artificial auditory tube, for such a tube, when it is inserted through the tympanic membrane, and then through the tympanic cavity until it gets at least up to the isthmus of the eustachian tube and is placed facing the inside of the cartilaginous eustachian tube, with the opening in the wall being located in the tympanic cavity, attains, in the case of stenosis of the eustachian tube (occlusion of the eustachian tube), nearly physiological levels of ventilatory and drainage functions through the eustachian tube by establishing communication between the nasal and the tympanic cavities, and, also in the case of patulous eustachian tube or closure defect of the eustachian tube, restores proper levels of communication between the nasal and tympanic cavities, through the tube's inner cavity, by at least partly clogging the over-expanded cross section of the isthmus of the eustachian tube. The present invention is completed by further investigation based on the finding.

Thus, the present invention provides:

(1) An artificial auditory tube consisting of a tubular body having a proximal end to be placed protruding out of the tympanic membrane within the external auditory canal and having a distal end to be inserted from the tympanic cavity into the eustachian tube and placed through the isthmus and facing the inside of the cartilaginous eustachian tube,

the distal end and the proximal end communicating with each other through an internal cavity extending in the axial direction of the tubular body,

the internal cavity opening to the outside through a first opening located at or close to the distal end, and

the external cross sectional dimension of the tubular body falling in the range of from 0.35 to 3.0 mm,

wherein the artificial auditory tube includes a second opening defined in the wall of the tubular body and positioned in the region ranging from 9 to 30 mm away from the distal end and communicating with the internal cavity of the tubular body.

(2) The artificial auditory tube of (1) above, wherein the cross sectional dimension of the internal cavity of the tubular body is not less than 0.20 mm and, at least partly in a region, not more than 0.9 mm.

(3) The artificial auditory tube of (1) or (2) above, wherein the artificial auditory tube includes a third opening defined in the wall of the tubular body and positioned in the region that is located between 1 and 16 mm away from the distal end and between 8 and 26 mm toward the distal end away from the second opening, the third opening communicating with the internal cavity of the tubular body.

(4) The artificial auditory tube of one of (1) to (3) above, wherein the tubular body comprises a shaft portion that is a relatively thick tubular portion including a proximal end region to be placed protruding out of the tympanic membrane within the external auditory canal and a distal portion that is a relatively thin tubular portion extending forward in the axial direction from the shaft portion to be inserted from the tympanic cavity into the eustachian tube through the tympanic opening of the eustachian tube to place the distal end through the isthmus facing the inside of the cartilaginous eustachian tube, and

the distal portion forms up to 4-20 mm from the distal end of the artificial auditory tube.

(5) The artificial auditory tube of (4) above, wherein the distal portion stepwise or continuously decreases in the external cross sectional dimension thereof along the direction from the proximal end to the distal end thereof relative to the shaft portion.

(6) The artificial auditory tube of (4) or (5) above, wherein the cross sectional dimension of the internal cavity of the shaft portion of the tubular body is expanded relative to the cross sectional dimension of the internal cavity of the distal portion of the tubular body.

(7) The artificial auditory tube of one of (4) to (6) above, wherein the external cross sectional dimension of the artificial auditory tube falls in the range of from 0.35 to 1.7 mm at the position where the first opening is located.

(8) The artificial auditory tube of one of (4) to (7) above, wherein the distal portion is made of two or more tubular portions differing in external cross sectional dimension and serially combined and aligned from the distal extremity of the shaft portion toward the distal end of the artificial auditory tube, and wherein the closer is a tubular portion to the distal end, the smaller is the external cross sectional dimension thereof.

(9) The artificial auditory tube of one of (4) to (8) above, wherein the distal portion consists of a forefront tubular portion and an intermediate tubular portion located between the forefront tubular portion and the shaft portion.

(10) The artificial auditory tube of (9) above, wherein the external cross sectional dimension of the intermediate tubular portion differs by at lease 0.15 mm from that of the forefront tubular portion and also from that of the shaft portion.

(11) The artificial auditory tube of (9) or (10) above, wherein the ratio in length of the forefront tubular portion to the intermediate tubular portion is 1:2 to 2:1.

(12) The artificial auditory tube of one of (1) to (11) above, wherein the full length thereof is 20 to 70 mm.

(13) The artificial auditory tube of one of (1) to (12) above which is made of a flexible material.

(14) The artificial auditory tube of one of (1) to (13) above, wherein the flexible material is a synthetic resin.

### THE EFFECT OF THE INVENTION

The present invention as respectively defined above serves, by properly restoring the ventilatory and drainage functions of the eustachian tube, to provide the basic treatment of a variety of middle ear disorders caused by eustachian tube dysfunction in patients with stenosis of the eustachian tube (occlusion of the eustachian tube), patulous eustachian tube, or closure defect of the eustachian tube, and also in patients having a “floppy tube”.

### BRIEF DESCRIPTION OF DRAWINGS

FIG. 1 illustrates a side view of the artificial auditory tube of Example 1.

FIG. 2 illustrates a side view of a variant of the artificial auditory tube of Example 1 having a rear portion with a reduced external cross sectional dimension.

FIG. 3 illustrates a side view of the artificial auditory tube of Example 2.

FIG. 4 illustrates a side view of the artificial auditory tube of Example 3.

FIG. 5 is a schematic illustration showing the principles of eustachian tube functions test based on the inflation-deflation method.

FIG. 6 is schematic illustration showing the principles of sonotubometry.

FIG. 7 is a chart showing the result of the test based on the inflation-deflation method of case 1 before treatment.

FIG. 8 is a chart showing the result of the test based on the inflation-deflation method of case 1 after treatment.

FIG. 9 is a chart showing the result of the test by sonotubometry of the case 1 after treatment.

FIG. 10 is a chart showing the result of the test based on the inflation-deflation method of case 2 after treatment.

FIG. 11 is a chart showing the result of the test based on the inflation-deflation method of case 2 after treatment FIG. 12 is a chart showing the result of the test based on the inflation-deflation method of case 3 before treatment.

FIG. 13 is a chart showing the result of the test based on the inflation-deflation method of case 3 after treatment.

FIG. 14 is a chart showing the result of the test based on the inflation-deflation method of case 3 after treatment.

FIG. 15 is a chart showing the result of the test based on the inflation-deflation method of case 4 before treatment.

FIG. 16 is a chart showing the result of the test based on the inflation-deflation method of case 4 after treatment.

### EXPLANATION OF THE SIGNS

1 artificial auditory tube
2 shaft portion
3 intermediate tubular portion
4 forefront tubular portion
5 distal end
6 proximal end
7 opening
8 opening
9 opening
11 artificial auditory tube
21 artificial auditory tube
22 tubular member
25 distal end
26 proximal end
27 opening
30 narrowed part
41 pressure transducer
42 amplifier
43 recorder
51 sonotubometry/analysis instrument

### BEST MODE FOR CARRYING OUT THE INVENTION

In the artificial auditory tube of the present invention, the second opening has as its primary objective to provide communication between the tympanic cavity and the cartilaginous eustachian tube of the patient through the inner cavity of the artificial auditory tube thereby to ensure ventilation between the tympanic and nasal cavities. Therefore, the artificial auditory tube is inserted in a patient in such a manner that the distal end portion of the artificial auditory tube face the inside of the cartilaginous eustachian tube through the isthmus to let the first opening, which is located at or close to the distal end, open to the inside of the cartilaginous eustachian tube and that the second opening, which is away from the first one, be located within the tympanic cavity. Accordingly, of artificial auditory tubes of the present invention, a tube of a proper size and having those openings at proper positions may be selected and used by a doctor, corresponding to the length and width of the patient's eustachian tube.

For example, in stenosis of the eustachian tube (occlusion of the eustachian tube), there are found some cases in which isthmus of the eustachian tube is so narrow that only a thin artificial auditory tube can be inserted without force that has an external cross sectional dimension of at most about 0.35 mm in its distal end portion, while there are other cases in which a relatively thicker artificial auditory tube can be inserted having an external cross sectional dimension of, e.g., 0.5 mm, 0.8 mm, 1.0 mm, etc. in its distal end portion. Moreover, in closure defect of the eustachian tube or patulous eustachian tube, some patients have the isthmus of the eustachian tube so expanded that it allows insertion without force of a tube having an external cross sectional dimension of about 3 mm in its distal end portion. As the width of the internal cavity of the isthmus of the eustachian tube (when forcibly expanded) can be almost precisely estimated from CT-images of the area of interest, a tube of a proper thickness suitable to a given patient may be chosen as desired from artificial auditory tubes varying in the external cross sectional dimension, i.e., 0.35-3.0 mm, in their distal end portion where the first opening is defined, e.g., 0.35 mm, 0.5 mm. 0.8 mm, 1.0 mm, 2.0 mm, 2.5 mm, 3.0 mm, etc. Where the external cross sectional dimension is made smaller in and near the distal end portion than the remaining part, the external cross sectional dimension of the distal end portion may be 0.35-1.7 mm.

The first opening in the distal end portion of the artificial auditory tube of the present invention may be an opening defined by the lumen just running through the very end of the tubular distal end portion in the axial direction. Contrarily, the lumen may be closed in the axial direction at the distal end but an opening may be defined laterally in the portion. A tube of the latter configuration is easy to handle when inserting the artificial auditory tube, by the help of a guide wire placed in its lumen, into the eustachian tube, for the guide wire will be stopped by the internal wall of the distal end of the artificial auditory tube and thus will not forwardly stick out of the tube even if the guide wire is thinner than the internal cross sectional dimension of the distal end portion.

The artificial auditory tube of the present invention is inserted usually following the procedure that: a proper guide wire is inserted in the inner cavity of the artificial auditory tube through its proximal end, and, while supported by the guide wire, it is inserted into the eustachian tube, and then left alone in the ear, with the guide wire only pulled out. Therefore, when inserting, it is preferred that the artificial auditory tube has an open proximal end through which the lumen just runs. However, in the case of an artificial auditory tube made of such a relatively stiff material that can support itself during the manipulation for insertion, as it does not require a guide wire, its proximal end may be closed. Thus, the artificial auditory tube of the present invention also encompasses those with closed proximal end.

When inserted into the eustachian tube of a patient, it is preferable that the artificial auditory tube of the present invention, at its distal end, reaches the isthmus of the eustachian tube or extends further beyond it. When it is inserted up to the isthmus of the eustachian tube, the first opening of the artificial auditory tube must be open to the inside of the cartilaginous eustachian tube. The artificial auditory tube is preferably inserted so that its distal end protrudes beyond the isthmus of the eustachian tube by at least 1 mm or at least 2 mm, since this allows more stable retention of the distal end portion of the artificial auditory tube within the isthmus, thus serving to eliminate the risk of displacement (slight retraction) of the artificial auditory tube that would lead to blockade of the first opening at the distal end by the isthmus. It is also preferred that the distal end of the artificial auditory tube is not positioned over 12 mm beyond the isthmus of the eustachian tube. This is because, by not placing the distal end of the artificial auditory tube too close to the pharyngeal orifice of the eustachian tube, it becomes easier to prevent contaminants in the nasal cavity from ascending.

The artificial auditory tube of the present invention is inserted through the tympanic cavity to bring its distal end up to the isthmus or to a position beyond it. Even when the distal end is inserted further beyond the isthmus, however, it is proper that the distal end stays within approximately 12 mm from the isthmus. When inserted in this manner, the first opening of the artificial auditory tube opens in the cartilaginous eustachian tube, thereby bringing the lumen of the artificial auditory tube in communication with the cartilaginous eustachian tube (and with the nasal cavity), while the second opening defined in the wall of the artificial auditory tube rests in the tympanic cavity. Thus, the communication between the tympanic cavity and the nasal cavity is attained. This communication not only functions to eliminate pressure difference between the tympanic cavity and the ambient atmosphere (i.e., ventilatory function) but also to drain secreted fluid in the tympanic cavity, if present, out into the nasal cavity (i.e., drainage function). As the length of the eustachian tube differs from patient to patient, especially between an adult and a child, and so is the length between the isthmus and the tympanic cavity, one may chose to use a proper size of an artificial auditory tube of the present invention which, when inserted, will have the second opening rest within the tympanic cavity, based upon the known length of the eustachian tube of a given patient determined by some means such as, e.g., CT-imaging, and in accordance with how deep to insert the artificial auditory tube beyond the isthmus. It is preferable that plurality of artificial auditory tubes have been provided in advance that have the second opening at a varying position in the range of from 9-30 mm from the distal end, for it enables to address patients with the eustachian tubes of varying sizes. While there is no specific limitation as to the size of the second opening, and it may be of a size that will not hinder the elimination of pressure difference between the tympanic cavity and the nasal cavity through the artificial auditory tube, it is preferable that the second opening is larger than the first opening defined in the distal portion, thereby allowing secreted fluid in the tympanic cavity, if present, to easily flow, through the second opening, in the artificial auditory tube and then flow down along it. Even when secreted fluid has entered the artificial auditory tube, no substantial pressure difference will thereby be created between the tympanic cavity and the nasal cavity (thus, the ventilatory function is maintained), since the secreted fluid will just go up and down in accordance with any pressure fluctuation between the tympanic cavity and the nasal cavity.

The cross sectional dimension of the lumen of the artificial auditory tube of the present invention is preferably not less than 0.20 mm. This is because a lumen with too narrow a cross sectional dimension could create resistance against the flow of air (and secreted fluid also, as the case may be) through it, while such a concern is substantially small if the cross sectional dimension is 0.20 mm or over. Too wide a lumen of the artificial auditory tube, contrarily, could bring about air-borne transmission of patient's own voice to the tympanic cavity, but this can be prevented by narrowing, partly, i.e., at least at some position, the cross sectional dimension of the flow path between the first and second openings of the artificial auditory tube. In such a case, the lower limit of the cross sectional dimension of the narrowed part of the flow path may be 0.20, and the upper limit of it is preferably 0.9 mm, and more preferably 0.8 mm. By so doing, the narrowed part provided in the flow path functions as an isthmus and prevents the patient's own voice from being air-borne to the tympanic cavity, even where the remaining part of the flow path has wider cross sectional dimensions.

There is no specific limitation as to the shape of the cross section of the artificial auditory tube of the present invention. It is usually preferable that the shape is circular or oval. The shape of the cross section may be either the same all along the length of the artificial auditory tube, e.g., circular all along the tube, or it may vary, e.g., circular in most of its length and oval in part (e.g., in the distal end portion), or oval in most of its length and circular in part (e.g., in the distal end portion). Where the shape is oval, the ratio in length of the major axis to the minor axis may be, at most, up to 4. As the cross section of the isthmus of the eustachian tube is of a flattened shape, being wider in the anteroposterior direction than in the lateral direction, if the distal portion of an artificial auditory tube has an oval cross section, it is easily fit to the most part of the entire inner circumference of the isthmus, therefore advantageous in the case of patulous eustachian tube for effectively clog the excess width of the lumen of the isthmus. Further, in the case of stenosis of the eustachian tube, an artificial auditory tube having approximately circular cross section in its distal portion is useful in ensuring a flow path for draining the secreted fluid in the tympanic cavity, because such an artificial auditory tube, when inserted into the isthmus, will somewhat expand forcibly the region of the inner circumference of the flattened isthmus not in direct contact with the outer surface of the artificial auditory tube's distal end portion, thus creating a small gap between the outer wall of the artificial auditory tube's distal end portion and the inner wall of the isthmus of the eustachian tube. Therefore, a doctor in charge may decide which artificial auditory tube of the present invention is preferred to be chosen as to the shape of its cross section, in accordance with the shape and the condition of the eustachian tube of each patient.

In the present invention, the term “cross sectional dimension” as mentioned concerning the artificial auditory tube means the diameter if the shape of the cross section (the outer circumference of the cross section, or the inner circumference, i.e., the circumference of the inner cavity) of the artificial auditory tube is circular, and the length of the minor axis if elliptical. Likewise, the term “cross sectional dimension” as mentioned concerning each of the openings in the artificial auditory tube means the diameter when the opening is circular, and the length (span) of the minor axis when the opening is of an oblong shape, e.g., elliptical or oval.

The artificial auditory tube of the present invention may be provided with a third opening that serves, when effusion comes in its lumen from the second opening, as a drain opening for quickly eliminating the effusion out of its lumen and into the nasal cavity. As already mentioned, the isthmus of the eustachian tube is, by nature, of a flattened cross sectional shape, and therefore, when an artificial auditory tube is inserted in that site, a gap is created in the isthmus around the artificial auditory tube as a result of forcible expansion of the isthmus by the inserted artificial auditory tube, and this gap provides a flow path for draining the effusion, unless the artificial auditory tube is forcibly inserted so that it completely fits the entire circumference of the cross section of the isthmus. By positioning the third opening in the artificial auditory tube so as to be located at that site or immediately upstream of it, the effusion that has entered the internal cavity of the artificial auditory tube flows out of the tube through the third opening according to its own weight and by the help of the surface tension, thus easily flow down through the gap, which is created in the isthmus due to its forced expansion by the artificial auditory tube, to the cartilaginous eustachian tube and then to the nasal cavity. The third opening may be of an elongated (e.g. slit-like) shape so that part of it can be positioned at or immediately above the isthmus and from there it extends downwardly beyond the isthmus. In this case, still better effects are achieved, for flowing down of effusion as well as ventilation are both promoted by being coupled with swallowing motions.

As the purpose of providing the third opening is as mentioned above, it is preferred that the opening is positioned such that it comes at or immediately above the isthmus, or, instead, it extends from such a position downwardly beyond the isthmus, when the artificial auditory tube is properly inserted in the eustachian tube of a patient. In order that an artificial auditory tube can be inserted up to a depth suitable to the condition of each of diverse patients with different length of the eustachian tube while ensuring that the third opening come at the aforementioned position, it is preferable that a set of artificial auditory tubes are provided in advance that have the third opening at varying position falling in the range of from 1 to 16 mm, e.g., from 1 to 15 mm, from the distal end. By this, when the distal end is to be positioned at the isthmus of the eustachian tube (in this case, usually, those having the first opening that opens forwardly are used), an artificial auditory tube may be chosen to use that has the third opening at a position falling in the range of from, e.g., 1 to 3 mm from the distal end, and when the distal end of an artificial auditory tube is to be positioned up to 12 mm beyond the isthmus, an artificial auditory tube may be used having the third opening at somewhere in the range of from 8 to 16 mm, or, e.g., from 13 to 16 mm from the distal end. However, as aforementioned is the purpose of the third opening, it is positioned distally as compared with the second opening. The position of the third opening is chosen in accordance with the size of the patient's eustachian tube to be treated and the intended depth up to which an artificial auditory tube is about to be inserted. By providing in advance artificial auditory tubes having the third opening at different positions falling in the above-described range and also falling in the range of from 8 to 26 mm away from the second opening in the distal direction, e.g., from 8 to 20 mm, a doctor in charge can choose and use a proper one suitable to a given patient.

Although an artificial auditory tube of the present invention may be of uniform thickness, this is not essential and, for example, the distal portion may be thinner than the proximal portion. With such configuration, there are cases in which easy handling is allowed during insertion, especially in patients with narrow isthmus, for the insertion can be made while supporting the distal, relatively thinner portion (distal portion) with the relatively thicker portion (shaft portion) closer to the proximal end. And, in order to reduce, by insertion of an artificial auditory tube, the effective cross sectional area of the isthmus with an expanded cross sectional dimension of a patient with patulous eustachian tube, the artificial auditory tube, for example, may have a sufficient thickness from the part located in the tympanic cavity through the part placed in and sandwiched by the isthmus, since the thickness of the inserted tube should match the isthmus, and have a still distal part with narrowed cross sectional dimensions. Such a configuration will allow easy insertion into the isthmus. When a distal portion and a shaft portion are provided, it is usually preferred that the distal portion extends up to 4-20 mm from the distal end of the artificial auditory tube. By providing the distal portion in such a range and making the remaining part as a shaft portion, it is possible, even in a tube for a patient having a narrow eustachian tube, to define the second opening in the relatively thicker shaft portion.

The shaft portion may be of uniform thickness. However it may also have a smaller external cross-sectional dimension in a region of the shaft portion that is to protrude out of the tympanic membrane (referred to the “proximal end region” in the present specification) than the external cross-sectional dimension of the remaining region of the shaft portion located relatively distally and placed completely within the tympanic cavity (the body of the shaft portion). This configuration is preferable, for it allows reduced cross-sectional area of a part of the artificial auditory tube protruding out of the tympanic membrane, without impairing easy insertion of the tube. The proximal end region may be either integrally molded with the body of the shaft portion or may be fit later in the proximal end of a molded body of the shaft portion.

There is no specific limitation regarding how to give a reduced external cross-sectional dimension to the distal portion extending distally from the shaft portion: it may have an external cross-sectional dimension continuously (e.g., gradually, lineally, etc.) reduced away from its starting point, or it may consist of one and single tubular portion thinner than the shaft portion, or it may consist of a plurality of connected tubular portions that successively reduce their external cross-sectional dimensions.

When the distal portion and the shaft portion are provided, the cross-sectional dimension of the lumen of the shaft portion may be made greater than the cross-sectional dimension of the lumen of the distal portion. Such configuration is advantageous to downwardly send and drain effusion that has entered the lumen.

The first opening is positioned at or near the distal end of the distal portion. The external cross-sectional dimension of this part of the artificial auditory tube may be 0.35-3.0 mm, and, for the majority of patients, preferably about 0.35-1.7 mm. By providing several types having different external cross-sectional dimensions within this range, such as 0.35 mm, 0.5 mm, 0.8 mm, 1.0 mm, etc., one can choose to use a proper artificial auditory tube suitable to insert into the isthmus and expand it which is narrowed at varying levels in patients having stenosis of the eustachian tube. For patients with patulous eustachian tube, it is sufficient to provide in advance artificial auditory tubes with greater cross-sectional dimensions.

The artificial auditory tube may be made so that, for example, the distal portion consists of two or more portions, in such a manner that, starting with the most distally located tubular portion, which is the thinnest, tubular portions are successively combined while increasing their thickness and that the last one of the tubular portions is combined with the shaft portion. That is, for example, the distal portion may be divided into two parts consisting of the distal tubular portion, which is the thinnest, and the intermediate tubular portion with intermediate thickness, which is combined with the former, with the intermediate tubular portion in turn combining itself with the shaft portion, which is the thickest. When this configuration is employed, the difference in external cross-sectional dimension between two adjoining tubular portions is usually not less than 0.15 mm. And, where, as in this case, the distal portion is made of a plurality of combined tubular portions of different thickness, the outer edge of the thicker one of tubular portions at each binding site is preferably chamfered or rounded in order to remove excess resistance against insertion. Where the distal portion is divided into two portions, easy handling during insertion into the eustachian tube will be provided if the ratio in length of the distal tubular portion to the intermediate tubular portion falls in the range of 1:2 to 2:1. Though the full length of the artificial auditory tube may be determined as desired, usually it is preferably not less than 20 mm even considering the cases of children, and, for easier handling, not more than 70 mm also considering cases of adults, and it also may be 60 mm or less, or 50 mm or less, too. However, one can cut off the proximal end as desired in use (prior to or following insertion into the eustachian tube) no problem will arise even if the full length is made longer than 70 mm.

As for the materials of which to form the artificial auditory tube of the present invention, such materials are preferred that are biocompatible, i.e. free of concern about triggering a harmful foreign-body reaction, and free of concern about undergoing decomposition or degradation in the body, and also flexible. As such, those materials that have so far been used in the medical field for devices to be implanted or indwelled in the body may be used as desired in preparing the artificial auditory tube. Examples of flexible synthetic resins include, but are no limited to, polyvinyl chloride, silicone, polyethylene, polypropylene, polypentene, and polyurethane based resins and the like. Such a resin that is designed to soften when warmed up to the body temperature is more preferable, for while it is easy to handle during insertion since it keeps proper stiffness, it will, after inserted, become softened due to the body temperature, thus being free of concern about giving foreign body sensation to the patient.

When inserted in a patient, the proximal end of the artificial auditory tube of the present invention is placed out of the tympanic membrane. In order to ensure stability so that the artificial auditory tube placed in the eustachian tube is prevented from back or forth displacement, it is sufficient that a tympanostomy tube commonly used in the field of otorhinolaryngology is fit around the artificial auditory tube and then the tympanostomy tube is fit in the perforation formed in the tympanic membrane as usual. By properly choosing the inner diameter of the tympanostomy tube and the external cross-sectional dimension of the shaft portion of the artificial auditory tube to be used, the artificial auditory tube can be set in place so that spontaneous displacement is prevented. When set in place by this way, spontaneous falling off of the tympanostomy tube, which is usually observed when it is set alone, becomes less likely to happen. Thus, it becomes possible to continue to have an artificial auditory tube stably fit in place over an extended period of time. The lumen of the artificial auditory tube may be open at its proximal end to be placed out of the tympanic membrane. However, closure of it by some proper means, such as fusion sealing, plugging with an ointment or he like, at some proper point of time after operation, will add to the clinical value as being more suitable artificial auditory tube. This is because, when such measures are taken, and if the artificial auditory tube and the tympanostomy tube are tightly fit and the perforation in the tympanic membrane has healed to closely surround the entire outer circumference of the tympanostomy tube, the air communication between the external auditory canal and the middle ear becomes blocked, and thus the middle ear is protected from direct contact with the ambient air, making the oxygen partial pressure in the tympanic cavity closer to the physiologic condition, which is beneficial to the health of the mucous membranes in the middle ear. Therefore, in the case where the artificial auditory tube is to be placed over a long period of time or for life, it is preferable to close the proximal end of it. However, follow-up observation of eustachian tube function is sometimes necessary several months after operation, and it is more convenient for examination, during such a period, that the closure of the proximal end of the artificial auditory tube is reversible (e.g., by clogging with an ointment). And, if the stenosis of the eustachian tube is not severe, there are some cases where the proximal end of the artificial auditory tube need not to be closed, as it is removed after cure is obtained by insertion of the artificial auditory tube for a relatively short period of time (3-4 weeks).

The artificial auditory tube of the present invention is placed in the eustachian tube of a patient with eustachian tube dysfunction for a required length of time in order to restore impaired ventilatory and drainage functions of the eustachian tube. By this, a variety of middle ear disorders that are caused by eustachian tube dysfunction, including otitis media with effusion, and adhesive otitis media, etc., can very effectively be treated. The length of time over which to place an artificial auditory tube depends on the severity of the eustachian tube dysfunction and the rate of restoration of the intrinsic eustachian tube functions. After the eustachian tube dysfunction has been cured by improving the conditions of the middle ear into normal ones with the help of the placed artificial auditory tube, the artificial auditory tube may be removed. In such a case where the eustachian tube has been so severely damaged that neither substantial cure is expected nor the maintenance of ventilatory and drainage functions can be possible without the help of an artificial auditory tube, the treatment using an artificial auditory tube may be continued, e.g., for life.

### EXAMPLE 1

The present invention is described in further detail below with reference to some typical examples. However, it is not intended that the present invention be limited to those examples.

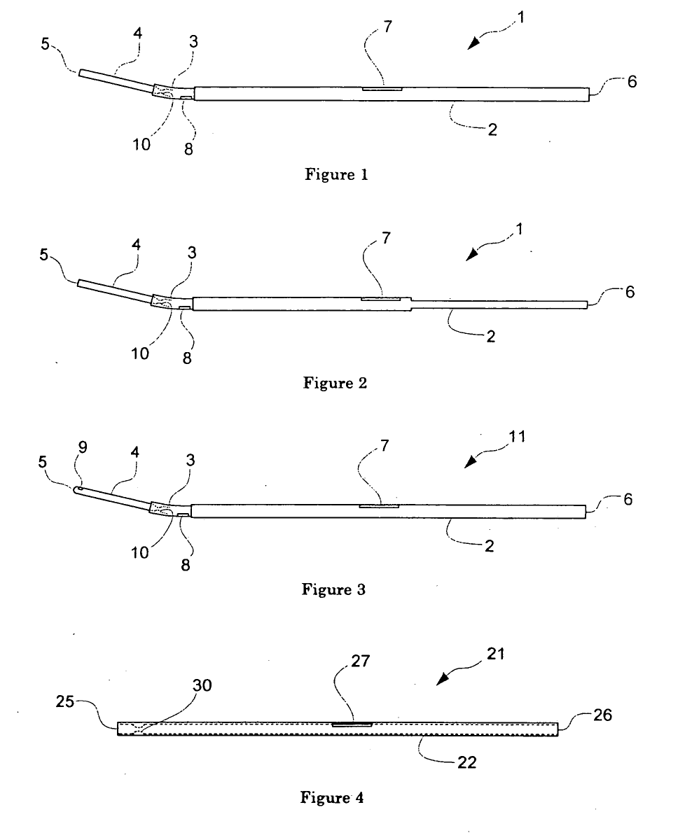

FIG. 1 illustrates a side view of Example 1 of the artificial auditory tube of the present invention. In the figure, the artificial auditory tube 1 consists of shaft a portion 2, which is a tubular portion having a relatively greater external cross-sectional dimension, an intermediate tubular portion 3, which also is a tubular portion but having a smaller external cross-sectional dimension than that of the former, and a distal tubular portion 4, which is a tubular portion having a still smaller external cross-sectional diameter. Any of these portions are circular in their cross sections. A lumen extends from the distal end to the proximal end of the artificial auditory tube 1, and the lumen is open to outside at the distal end 5 and the proximal end 6, respectively. In this example, the full length of the artificial auditory tube 1 is 47 mm, of which the length of the distal tubular portion is approximately 7 mm, and the intermediate tubular portion approximately 4 mm. The distal tubular portion 4 has the external diameter of approximately 0.6 mm and the internal diameter of approximately 0.4 mm, the intermediate tubular portion 3 has the external diameter of approximately 0.9 mm and the internal diameter of approximately 0.7 mm, and the shaft portion 2 has the external diameter of approximately 1.1 mm and the internal diameter of approximately 0.9 mm. Each of these portions is a tube made of polyurethane resin. In this example, a part of the intermediate tubular portion 3 is fit in the lumen of the shaft portion 2, and a part of the distal tubular portion is fit in the lumen of the intermediate tubular portion, and the portions are fusion bonded and unified at the sites where they are fit to form. An opening 7 is provided in the tubular wall of the shaft portion 2 at the position ranging of from approximately 26 mm to 29 mm away from the distal end. The opening 7 is intended to be located within the tympanic cavity when the artificial auditory tube 1 is inserted in the eustachian tube of a patient, and, when the artificial auditory tube is placed in such a manner, the opening 7 serves to achieve a ventilatory function chiefly in cooperation with the opening 5 at the distal end, which, through the isthmus, opens to the inside of the cartilaginous eustachian tube, and the lumen communicating with it. Thus, the air pressure in the tympanic cavity of a patient with stenosis of the eustachian tube (occlusion of the eustachian tube) can be made identical to that in the nasal cavity, even after the distal end of the artificial auditory tube 1 is closed.

The intermediate tubular potion 3 is provided with an opening 8 at a position approximately 9-10 mm from the distal end. In addition, at relatively distal position of the intermediate tubular portion 3, a narrowed part 10 with the internal diameter of 0.25 mm is defined in the internal cavity. The opening 8 is provided so that it should rest immediately above the isthmus when the artificial auditory tube 1 is inserted in the eustachian tube of a patient until its distal end extends beyond the isthmus of the eustachian tube by several millimeters, and it is intended to function as a drainage hole when effusion has entered the lumen. Thus, effusion that has entered between the opening 8 and the opening 7 flows, according to its own weight and by the effect of the surface tension of the effusion present externally around the opening 8, out of the opening 8 at immediately above of the isthmus, and then down through the flat gap somewhat expanded by the distal tubular portion 4 or the intermediate tubular portion 3, to the cartilaginous eustachian tube side and into the nasal cavity. Thus, in a patient with stenosis of the eustachian tube, the drainage function of the eustachian tube is assisted, thereby solving the problem of collected effusion in the tympanic cavity.

In the artificial auditory tube 1 of the present invention, the distal tubular portion 4 and the intermediate tubular portion 3, both forming the distal portion, are bended. The purpose of this is to imitate the curved shape of the eustachian tube so that the artificial auditory tube 1 can be inserted with ease into the eustachian tube through the tympanic membrane. It is also allowed not to give such curvature in advance, for it can be given as desired by a doctor in charge during operation. Furthermore, the shaft portion 2 may be narrowed in its external diameter in the proximal end region, which is to protrude out through the tympanic membrane as seen in FIG. 2 (the same applies to other examples).

When inserting the artificial auditory tube 1 of this example into the eustachian tube of a patient through the tympanic membrane, it is preferable that a guide wire of a proper diameter is inserted into the artificial auditory tube 1 through the proximal end 6 to add to its mechanical strength. Lest the tip of the guide wire stick out of the distal end of the artificial auditory tube 1, the diameter of the guide wire is preferably greater than that the internal diameter of the distal tubular portion 4. The artificial auditory tube 1, supported from within the lumen by the guide wire, is inserted through a perforation made in the tympanic membrane into the tympanic cavity, and through the tympanic opening of the eustachian tube up to an intended position of the eustachian tube of the patient, and the guide wire then is pulled out, leaving the artificial auditory tube 1 alone in the eustachian tube of the patient.

Then, around the shaft portion 2, i.e., the proximal portion of the artificial auditory tube 1 inserted in the patient's eustachian tube, a tympanostomy tube is fit having an internal diameter to fit the external diameter of the shaft portion 2. As the tympanostomy tubes of different sizes and shapes are commercially available, one may stock proper tubes in advance and choose and use one of them as desired. The perforation made in the tympanic membrane must be of the shape and size that will match the tympanostomy tube about to be used. The tympanostomy tube fit is then slid up to the position where it comes to intersect the tympanic membrane, and left there plugging the perforation in the tympanic membrane. Usually, the proximal end of the artificial auditory tube 1 is preferably occluded at a later proper time by fusion sealing, plugging with an ointment or any other proper means. In that state, when the tympanic membrane heals and the gap is closed between the tympanostomy tube and the tympanic membrane, the tympanic cavity is isolated from the external auditory canal.

### EXAMPLE 2

FIG. 3 illustrates a side view of Example 2 of the artificial auditory tube 11. This example differs from Example 1 of the auditory tube only at and near the distal end. Thus, in this example, the distal end 5 is closed and an opening 9 is defined laterally in the tubular wall. The opening 9, which is in communication with the lumen, performs the same function as the opening at the distal end of the Example 1 of the artificial auditory tube 1. However, in Example 2, since the distal end 5 of the artificial auditory tube 11 is closed, a guide wire, when inserted into the lumen through the proximal end 6 for insertion into the patient's eustachian tube, will not stick out of the distal end 5 even if the external diameter of the guide wire is smaller than the internal diameter of the distal tubular portion 4, for the tip of the guide wire will abut on and be stopped by the closed distal end 5 of the artificial auditory tube 11. Therefore, this adds to the degree of freedom of the doctor in choosing the external diameter of a guide wire, and thus contributes to the convenience in operation.

### EXAMPLE 3

FIG. 4 illustrates a side view of Example 3 of the artificial auditory tube 21. This example, which consists of a tubular member 22 of a uniform external diameter of approximately 2.5 mm having a lumen that is open at the distal end 25 and the proximal end 26, is for use in the treatment of patulous eustachian tube. It is linear in the figure, but its distal portion may be bent as in Example 1. Its tubular wall is provided with an opening 27, at a position approximately 18 to 21 mm from the distal end 25 of the artificial auditory tube 21. It has a narrowed part 30 that is formed by a circular inner projection of the inner wall at a certain part of the lumen, and internal diameter of the narrowed part 30 is approximately 0.35 mm. The artificial auditory tube of this example is intended to be inserted, with its distal end being placed in the isthmus of the eustachian tube and facing inside of the cartilaginous eustachian tube, for the purpose of reducing, by clogging, the effective cross sectional area of the expanded isthmus of a patient with patulous eustachian tube. The opening 27 is intended to perform the same function as the opening 7 in Example 1. The narrowed part 30 is a part corresponding to the isthmus, and due to its small inner diameter, blocks air-bone transmission of patient's own voice to the tympanic cavity, and, when needed, provide a downward flow path for effusion that has entered the internal cavity of the artificial auditory tube 21. The narrowed part 30 can also provide a stopper on which the tip of a guide wire abuts and is stopped, in the case where a guide wire is used for insertion of the artificial auditory tube 21. In this example, no drainage opening is provided as corresponding to the opening 8 in Example 1. However, even if effusion is present in the tympanic cavity, it will freely flow down through the flow path defined between the isthmus and the external surface of the artificial auditory tube 21 inserted in the flat isthmus, and thus will not be accumulated in the tympanic cavity. However, it is also allowed to provide an opening at a position somewhere proximal compared with the narrowed part 30. It is also allowed not to provide a narrowed part if the internal diameter is uniformly small, e.g., equal to or less than 0.9 mm, or equal to or particularly, less than 0.8 mm, for in such a case air-bone transmission of the patient's own voice hardly occurs. It is also allowed that the distal end 25 is closed as in Example 2 and an opening is laterally provided which corresponds to the opening 9 in Example 2. Further, though this example is a thick artificial auditory tube for use in patulous eustachian tube, thinner artificial auditory tube for use in stenosis of the eustachian tube (occlusion of the eustachian tube) may also be formed likewise. For this, however, the external diameter should be made to match the lumen of the isthmus (when forcibly expanded) of the patient, e.g., 0.4 mm, 0.6 mm, 1.0 mm, etc. and a drain opening (as corresponding to the opening 8 in Example 1) is preferably provided.

### EXAMPLE 4

[Clinical Test]

Part of results of clinical tests that were carried out in patients using artificial auditory tubes of the type described in Example 1 of the present invention is shown below.

(Test Method)

Eustachian tube functions test: The functional test of eustachian tube was carried out by the inflation-deflation method and sonotubometry.

Two methods are included in the test according to the inflation-deflation method, i.e. a static test, in which air pressure increasing at a constant rate is applied from the external auditory canal to the tympanic cavity through a perforation in the tympanic membrane, and the pressure is measured at which the eustachian tube spontaneously opens (referred to as “passive opening pressure” or “reverse aeration pressure”), and a dynamic test, in which, under a constant positive or negative pressure applied to the middle ear from the external auditory canal, the degree of opening of the eustachian tube by swallowing motion (active opening) is examined. According to the inflation-deflation test, when an increased pressure is applied to the tympanic cavity through the external auditory canal, restoration of the pressure by its abrupt reduction is observed in a normal eustachian tube, since the eustachian tube is opened by swallowing motion. Also, when the applied air pressure is gradually increased and it reaches a certain level, the eustachian tube is passively expanded to open (passive opening) and the air flows out, thus exhibiting pressure reduction at the point. In stenosis of the eustachian tube, in contrast, as the eustachian tube remains closed during swallowing, even repeated swallowing would fail to reduce the tympanic cavity pressure, and passive opening of the eustachian tube will not occur until the pressure in the tympanic cavity reaches significantly high levels. Passive opening pressure, on an average, is approximately 355 daPa in normal ears, and the pressure is regarded as normal when falling within two times the standard deviation, and thus a pressure over 545 daPa can be determined as being abnormal.

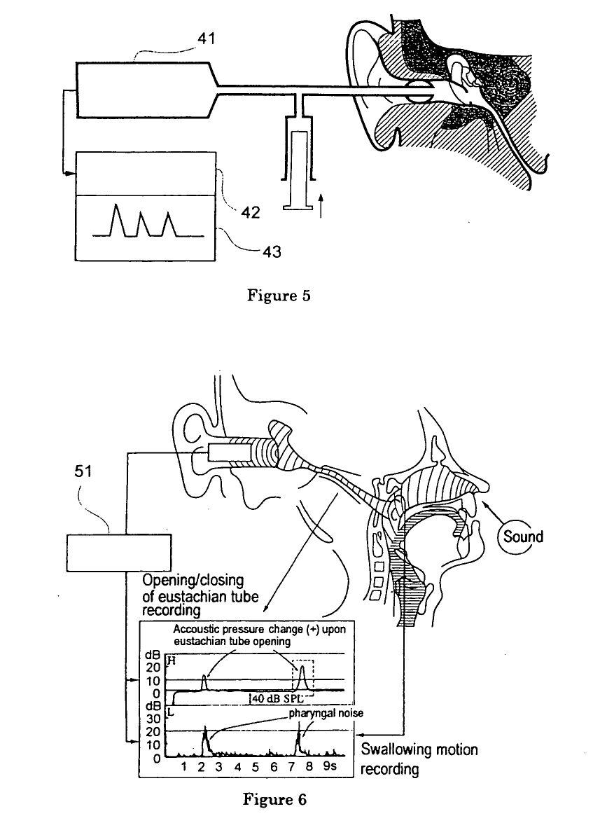

Examination by inflation-deflation method was conducted using a eustachian tube function testing instrument ET-1000 manufactured by NAGASHIMA MEDICAL INSTRUMENTS. CO., LTD (schematically illustrated in FIG. 5). The channel of the instrument was adjusted to the plot diagram creation by the inflation-deflation method, and air pressure (positive or negative) was loaded through the external auditory canal of a patient with a perforated tympanic membrane. Pressure changes when the eustachian tube opened (by swallowing or by increase in the loaded pressure) were measured by a pressure transducer 41, amplified by an amplifier 42, recorded in a recorder 43, and evaluated.

Sonotubometry is a test method in which the eustachian tube's condition is examined regarding its opening and closing by monitoring the acoustic pressure changes accompanied by swallowing motions by means of a microphone attached in the external auditory canal, while emitting into the nasal cavity a sound load from a sound generator. As the sound in the nasal cavity is air-bore through the eustachian tube to the external auditory canal if the eustachian tube opens during swallowing, the presence or absence and the degree of stenosis of the eustachian tube can be assessed based on the acoustic pressure changes detected by the microphone placed in the external auditory canal.

Examination by sonotubometry was conducted using a eustachian tube function testing instrument ET-1000 manufactured by NAGASHIMA MEDICAL INSTRUMENTS. CO., LTD (schematically illustrated in FIG. 6). The channel was adjusted to “sonotubometry” in the instrument, and assessment was made as to whether the eustachian tube opened during swallowing, by simultaneously monitoring swallowing motions and acoustic pressures in the external auditory canal.

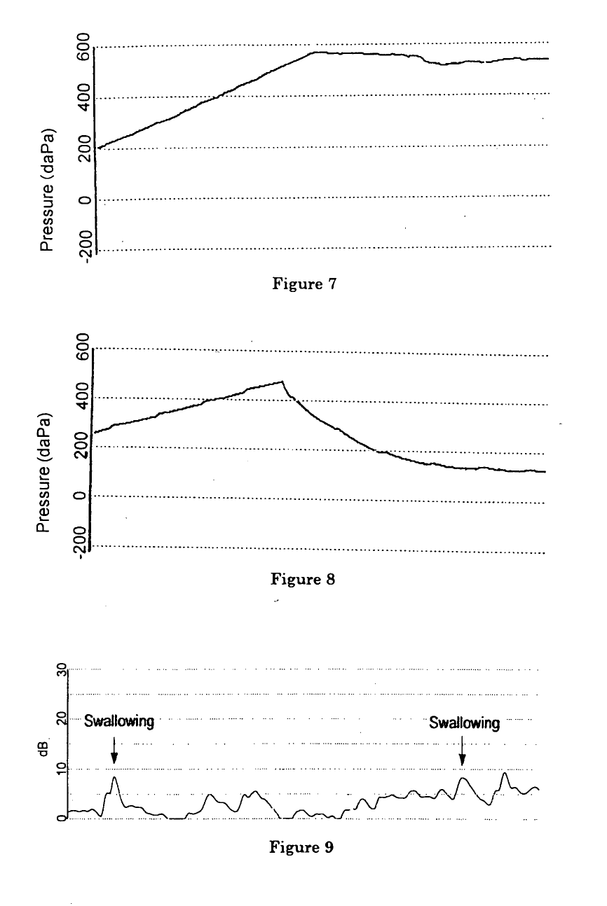

(Case 1) Using the inflation-deflation method, eustachian tube function prior to insertion of the artificial auditory tube was tested in a male patient, 72 years of age, having stenosis of the eustachian tube in his left ear. The result is shown in FIG. 7. The lower graph in FIG. 7 represents the pressure applied to the tympanic cavity, and the lower one occurrence of swallowing motions. As evident from the figure, even when the pressure load onto the external auditory canal was gradually elevated up to approximately 560 daPa, no opening of the eustachian tube took place, thus exhibiting a typical condition of stenosis of the eustachian tube. An artificial auditory tube as in Example 1, was inserted into the left ear of the patient through the tympanic membrane until the distal end of the tube reaches a point several mm beyond the isthmus of the eustachian tube. The artificial auditory tube was placed in a fixed position by fitting a tympanostomy tube around a part 10 mm away from the proximal end and also fitting the tympanostomy tube in the perforation in the tympanic membrane. For allowing reexamination by the inflation-deflation method, the proximal end of the artificial auditory tube was left open.

Two months after the insertion of the artificial auditory tube, reexamination was conducted by the inflation-deflation method, under the condition that the inserted artificial auditory tube was left untouched. The result is shown in FIG. 8. As seen in FIG. 8, it was observed that, after staring to load positive pressures to the tympanic cavity, the eustachian tube passively opened when the pressure reached approximately 470 daPa, and, at the same time, the pressure in the tympanic cavity rapidly reduced and returned to normal pressure, and thus confirming that the ventilatory function has been normalized between the tympanic and nasal cavities through the artificial auditory tube. By sonotubometry, as shown in FIG. 9, it was observed that the acoustic pressure load in the external auditory canal hiked during swallowing, i.e., the amount of the sound increased that was transmitted from the nasal cavity to the external auditory canal. Thus, this examination also confirmed that the eustachian tube function is normalized through the artificial auditory tube.

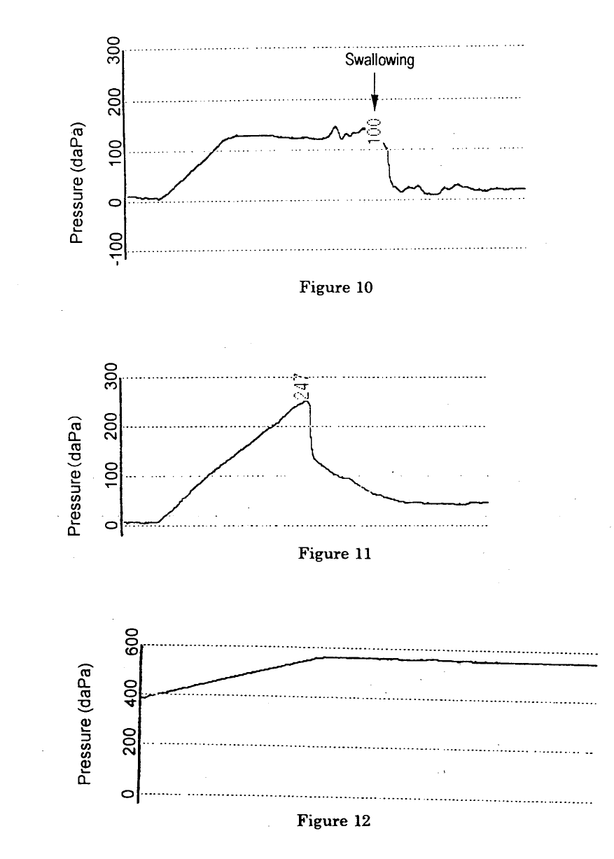

(Case 2) The therapeutic effect of the placement of an artificial auditory tube was assessed in a female patient, 41 years of age, with stenosis of the eustachian tube in her right ear. Prior to the insertion of an artificial auditory tube, the examination of eustachian tube function by inflation-deflation method revealed the typical pattern of stenosis of the eustachian tube as seen in Case 1. An artificial auditory tube as in Example 1 was inserted into the right ear of the patient through the tympanic membrane until its distal end reached a point several mm beyond the isthmus of the eustachian tube, and placed at a fixed position as in Case 1 using a tympanostomy tube. Three months after the operation, the artificial auditory tube was pulled out. Further three months later, eustachian tube function was examined. The result of the dynamic test is shown in FIG. 10. From the figure, it was confirmed that tympanic cavity pressure, which had been kept at approximately 130 daPa, returned to the ambient pressure due to the opening of the eustachian tube caused by a swallowing motion. In reverse aeration pressure test, as shown FIG. 11, the eustachian tube was passively opened when the air pressure was elevated up to 247 daPa, and the tympanic cavity pressure reduced almost to the ambient pressure. These results confirmed that the eustachian tube was normally functioning three months after removal of the artificial auditory tube, and that stenosis of the eustachian tube had cured.

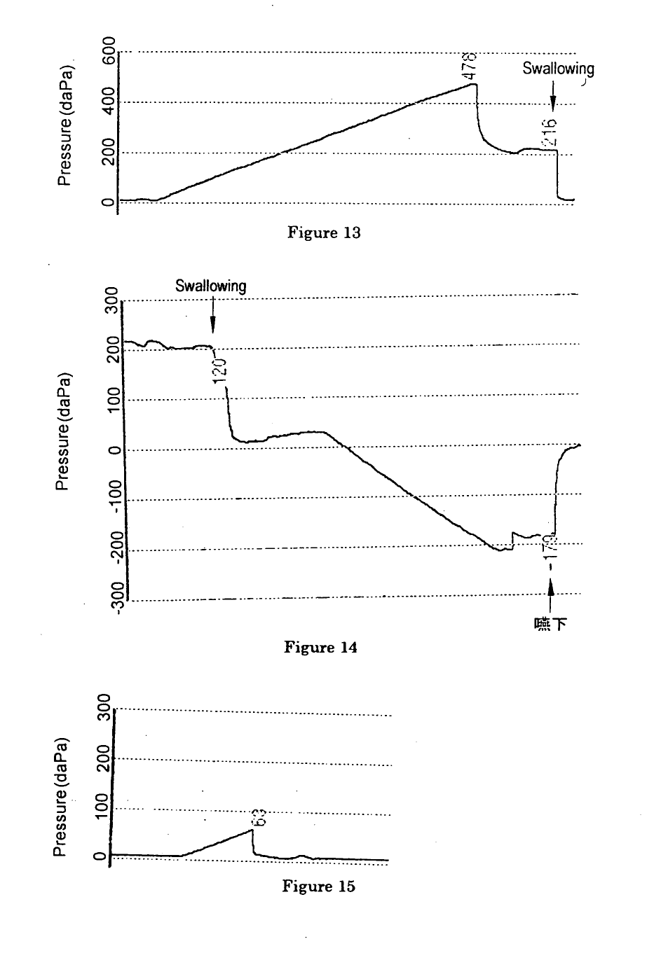

(Case 3) By the inflation-deflation method, eustachian tube function prior to insertion of the artificial auditory tube was tested in a female patient, 42 years of age, having stenosis of the eustachian tube (occlusion of the eustachian tube). As seen in FIG. 12 showing the results of examination using reverse aeration, no pressure reduction, therefore no opening of the eustachian tube, was observed even when the pressure was elevated up to approximately 570 daPa, and thus the presence of stenosis of the eustachian tube was confirmed. An artificial auditory tube as described in example 1 was inserted so that its distal end reached a point several mm beyond the isthmus of the eustachian tube and placed there. About two months later, the artificial auditory tube was pulled out, and eustachian tube function was tested. Examination by the reverse aeration method, as seen in FIG. 13, confirmed that the eustachian tube was passively opened at 478 daPa, and that it opened during swallowing at 216 daPa, allowing the tympanic cavity to return to the ambient pressure. By inflation-deflation test, in which negative pressure was also applied, it was confirmed that the eustachian tube opened during swallowing and the tympanic cavity returned to the ambient pressure, either under positive or negative pressure applied through the external auditory canal (FIG. 14). From these results, it was concluded that the eustachian tube functions of the patient has restored to normal.

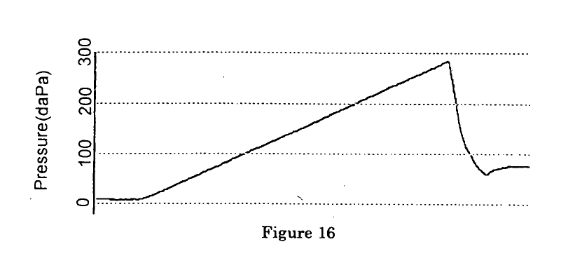

(Case 4) By the inflation-deflation method, eustachian tube functions prior to insertion of an artificial auditory tube was tested in a female patient, 39 years of age, with patulous eustachian tube. As a result, as seen in FIG. 15, passive opening of the eustachian tube was observed at the pressure of 63 daPa. An artificial auditory tube, which was the same type as described in example 1 but in which the external diameter of the portion corresponding to the shaft portion 2 was 1.7 mm, the external diameter of the portion corresponding to the intermediate tubular portion 3 was 1.3 mm, and the external diameter of the portion corresponding to the distal tubular portion 4 was 0.85 mm, was inserted until its distal end reached to a point several mm beyond the isthmus of the eustachian tube and placed there. Twenty days later, examination of eustachian tube function was conducted by inflation-deflation method, with the inserted artificial auditory tube being left untouched. As a result, as seen in FIG. 16, no opening of the eustachian tube occurred up to approximately 280 daPa, and thus it was confirmed that the patulous eustachian tube was properly corrected.

### INDUSTRIAL APPLICABILITY

The artificial auditory tube of the present invention is useful as an instrument that realizes a very effective treatment of eustachian tube dysfunction and a variety of middle ear disorders caused by it.

1. An artificial auditory tube consisting of a tubular body having a proximal end to be placed protruding out of the tympanic membrane within the external auditory canal and having a distal end to be inserted from the tympanic cavity into the eustachian tube and placed through the isthmus and facing the inside of the cartilaginous eustachian tube,
the distal end and the proximal end communicating with each other through an internal cavity extending in the axial direction of the tubular body,
the internal cavity opening to the outside through a first opening located at or close to the distal end, and
the external cross sectional dimension of the tubular body falling in the range of from 0.35 to 3.0 mm,
wherein the artificial auditory tube includes a second opening defined in the wall of the tubular body and positioned in the region ranging from 9 to 30 mm away from the distal end and communicating with the internal cavity of the tubular body.

2. The artificial auditory tube of claim 1, wherein the cross sectional dimension of the internal cavity of the tubular body is not less than 0.20 mm and, at least partly in a region, not more than 0.9 mm.

3. The artificial auditory tube of claim 1, wherein the artificial auditory tube includes a third opening defined in the wall of the tubular body and positioned in the region that is located between 1 and 16 mm away from the distal end and between 8 and 26 mm toward the distal end away from the second opening, the third opening communicating with the internal cavity of the tubular body.

4. The artificial auditory tube of claim 1, wherein the tubular body comprises a shaft portion that is a relatively thick tubular portion including a proximal end region to be placed protruding out of the tympanic membrane within the external auditory canal and a distal portion that is a relatively thin tubular portion extending forward in the axial direction from the shaft portion to be inserted from the tympanic cavity into the eustachian tube through the tympanic opening of the eustachian tube to place the distal end through the isthmus facing the inside of the cartilaginous eustachian tube, and
the distal portion forms up to 4-20 mm from the distal end of the artificial auditory tube.

5. The artificial auditory tube of claim 4, wherein the distal portion stepwise or continuously decreases in the external cross sectional dimension thereof along the direction from the proximal end to the distal end thereof relative to the shaft portion.

6. The artificial auditory tube of claim 4, wherein the cross sectional dimension of the internal cavity of the shaft portion of the tubular body is expanded relative to the cross sectional dimension of the internal cavity of the distal portion of the tubular body.

7. The artificial auditory tube of claim 4, wherein the external cross sectional dimension of the artificial auditory tube falls in the range of from 0.35 to 1.7 mm at the position where the first opening is located.

8. The artificial auditory tube of claim 4, wherein the distal portion is made of two or more tubular portions differing in external cross sectional dimension and serially combined and aligned from the distal extremity of the shaft portion toward the distal end of the artificial auditory tube, and wherein the closer is a tubular portion to the distal end, the smaller is the external cross sectional dimension thereof.

9. The artificial auditory tube of claim 4, wherein the distal portion consists of a forefront tubular portion and an intermediate tubular portion located between the forefront tubular portion and the shaft portion.

10. The artificial auditory tube of claim 9, wherein the external cross sectional dimension of the intermediate tubular portion differs by at lease 0.15 mm from that of the forefront tubular portion and also from that of the shaft portion.

11. The artificial auditory tube of claim 9, wherein the ratio in length of the forefront tubular portion to the intermediate tubular portion is 1:2 to 2:1.

12. The artificial auditory tube of claim 1, wherein the full length thereof is 20 to 70 mm.

13. The artificial auditory tube of claim 1 which is made of a flexible material.

14. The artificial auditory tube of claim 1, wherein the flexible material is a synthetic resin.
